(19) United States
(12) Patent Application Publication
(10) Pub. No.: US 2024/0165871 A1
(43) Pub. Date: May 23, 2024

(54) PROCESS FOR MANUFACTURING A MONOLITHIC PART

(71) Applicant: ANWERINA AG, Tägerwilen (CH)

(72) Inventors: Uwe SCHEITHAUER, Dresden (DE); Christoph KARL, Tägerwilen (CH)

(73) Assignee: ANWERINA AG, Tägerwilen (CH)

(21) Appl. No.: 18/552,832

(22) PCT Filed: Apr. 12, 2022
(86) PCT No.: PCT/EP2022/059684

§ 371 Date: Sep. 27, 2023

(30) Foreign Application Priority Data
Apr. 21, 2021 (DE) 10 2021 110 029.1

(51) Int. Cl. B29C 64/124; B29C 64/282; B29C 64/35; B29C 64/386; B29L 31/00; B33Y 10/00; B33Y 40/20; B33Y 50/00; B33Y 70/10; B33Y 80/00

(52) U.S. Cl. CPC B29C 64/124; B29C 64/282; B29C 64/35; B29C 64/386; B29L 2031/7532; B33Y 10/00; B33Y 40/20; B33Y 50/00; B33Y 70/10; B33Y 80/00

(57) ABSTRACT

The invention relates to a process for manufacturing a monolithic part, wherein different surface topologies are created in specifically selected regions in order to create different surface properties, in particular with respect to a wetting behavior, a deposition behavior and/or a colonization with cells.

The present invention relates to a process for forming a monolithic component and in particular to a process for forming a joint implant for tissue regeneration at the joint or a technical functional body using an additive lithography-based manufacturing technique.

Published patent application WO 2010/045950 A1 discloses a process for forming a monolithic component using a lithography-based manufacturing technique, wherein a photopolymerizable material is provided in a tray having a transparent tray bottom, a platform is immersed in the photopolymerizable material to a predetermined distance from the tray bottom, a selective irradiation through the tray bottom is performed to produce a polymerized layer body adherent to the platform which has a height corresponding to the distance, the platform comprising the polymerized layer body is vertically displaced to form a subsequent layer body and by repeating these layer production steps the monolithic component is built up layerwise in the desired shape.

It is necessary in many technical sectors to produce materials having different surface properties at different surface regions, for example wetting behaviour.

Wetting behaviour is hereinbelow to be understood as meaning the behaviour of liquids upon contact with a solid surface. Wettability is the accompanying property of the solid surface for a particular liquid and is often defined by the so-called contact angle of a liquid droplet on the surface of the solid. Wetting is nonexistent or only slight when the contact angle is in a range from 180° to 90°, with formation of a spherical or hemispherical liquid droplet which has only a small (or punctiform) contact area and easily glides over the solid surface (beading). Partial wetting is in effect when the contact angle is less than 90°, where a liquid droplet forms a round cap at the solid surface (runoff). Complete wetting is in effect when the contact angle is virtually 0°, where a liquid droplet spreads out in the shape of a flat sheet on the solid surface. In this case liquid residues remain adherent to the solid surface even under the influence of strong forces (for example extreme inclination of the surface).

In a special case of water as the liquid employed, reference is made in this context to hydrophobic (water-repellent) or hydrophilic (water-attracting) solid surfaces.

Both in the case of technical functional bodies (catalysts, capacitors, membranes . . . ) and in the case of materials and bodies used in medical technology different wetting behaviour is often desired and has hitherto needed to be realized through time intensive and costly aftertreatment processes (mechanical/chemical aftertreatment and/or chemical coating) after production of the starting material.

The present invention accordingly has for its object to provide a process for producing a monolithic component, wherein different surface properties, in particular in terms of wetting behaviour, deposition behaviour and/or colonization with cells may be realized locally in simple and cost-effective fashion.

According to the invention this object is achieved by the features of claim 1.

Especially the forming of different surface topologies in specifically selected regions of the monolithic component makes it possible to produce in these regions different surface properties, in particular in terms of wetting behaviour, deposition behaviour and/or colonization with cells.

The monolithic component is preferably produced from a design component (CAD or CAM) having a multiplicity of design elements, wherein at least one design orientation of a design element relative to a build space orientation of an actual build space is specifically altered.

The forming of the monolithic component is carried out for example using an additive manufacturing technique, wherein a photopolymerizable material is provided in a tray having a transparent horizontal tray bottom, a platform is immersed in the photopolymerizable material to a predetermined distance from the tray bottom, a selective irradiation through the tray bottom is performed to produce a polymerized layer body adherent to the platform which has a height corresponding to the distance, the platform comprising the polymerized layer body is vertically displaced to form a subsequent layer body and by repeating the preceding layer production steps the monolithic component is built up layerwise in the desired shape. Adjusting manufacturing parameters during the repeating of the layer production steps produces different surface topologies for realizing the different surface properties in selected regions of the monolithic component.

A component produced using this lithography-based 3D printing technique may be produced in a particularly cost-effective manner since aftertreatment not only of a green body but also of the finished sintered ceramic is thus rendered unnecessary and is also made possible at inaccessible sites.

As a manufacturing parameter for example an irradiation parameter, in particular an irradiation intensity and/or an irradiation time, may in the selective irradiation through the tray bottom be adjusted such that in addition to the layer body to be polymerized photopolymerizable residual material remaining in the edges and/or corners of the stepped structure is polymerized/crosslinked.

This allows simultaneous formation of different surface topologies on opposite surface regions in particularly simple fashion.

As a manufacturing parameter it is further possible to alter a rotation angle between horizontal design element principal axes and horizontal build space principal axes or a row or column alignment of a micromirror unit.

This allows different surface topologies to be formed on the lateral surfaces for each polymerized layer body.

As a manufacturing parameter it is further possible to alter a tilting angle between a vertical design element principal axis and a vertical build space principal axis/a perpendicular to the horizontal tray bottom.

This makes it possible to form different surface topologies in the vertical direction.

As a manufacturing parameter it is further possible to alter the distance between the platform/the last polymerized layer body and the tray bottom during the repeating of the layer production steps.

This further makes it possible to form different surface topologies in the vertical direction.

As a manufacturing parameter it is further possible to alter the dimensions of the pixels reflected by the micromirrors of the micromirror unit via an imaging unit during the repeating of the layer production steps.

This makes it possible to further alter the surface topologies.

The further subsidiary claims characterize further advantageous embodiments of the invention.

The invention is more particularly elucidated hereinbelow with the aid of exemplary embodiments and with reference to the drawing.

In the figures:

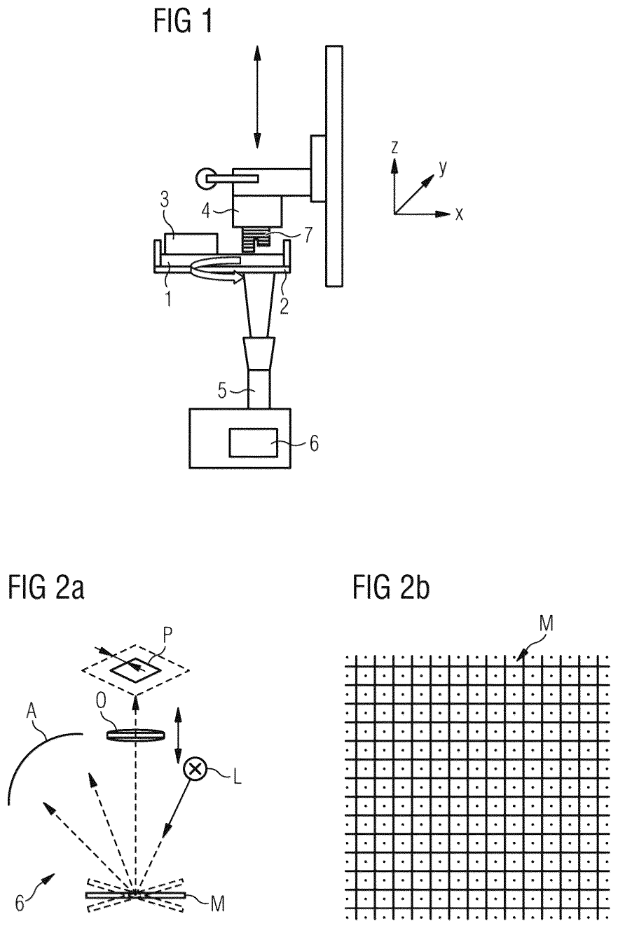

FIG. 1 shows a simplified side view of an apparatus for layerwise buildup of a monolithic component;

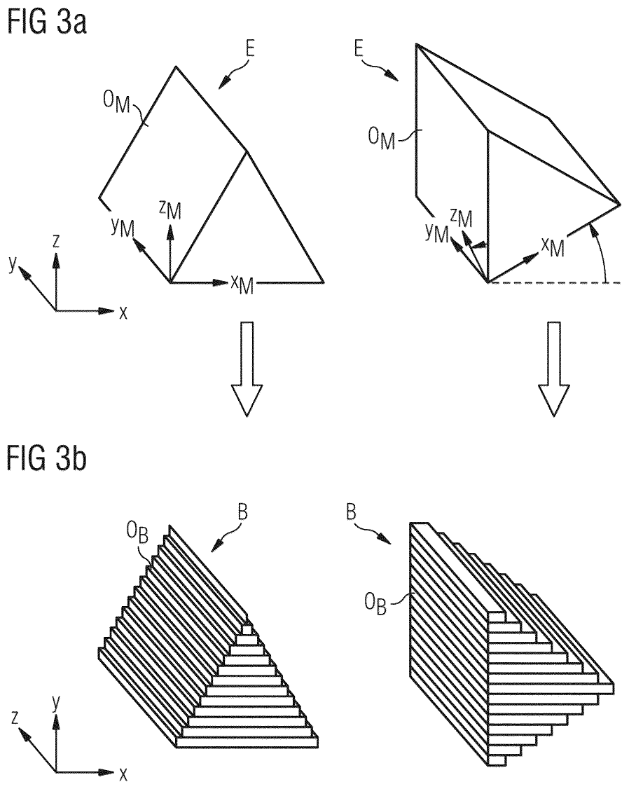

FIGS. 2a and 2b show simplified views of a micromirror unit employed;

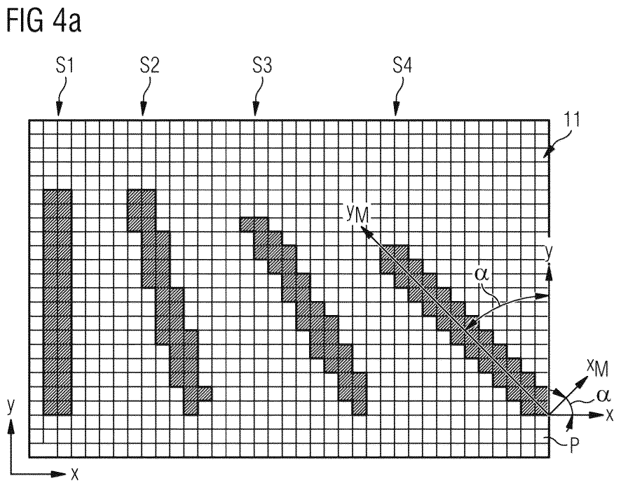

FIGS. 3a and 3b show simplified perspective views for elucidation of process steps according to a first exemplary embodiment, wherein different surface topologies are formed as a result of a deliberate alteration of a design orientation of a design element relative to a build space orientation;

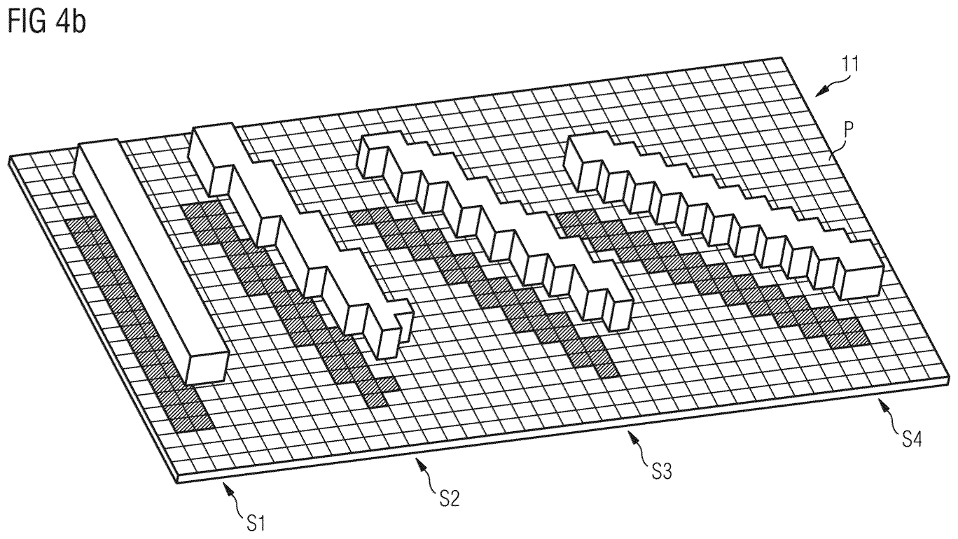

FIGS. 4a to 4c show simplified views for elucidation of process steps in the selective production of different surface topologies according to a second exemplary embodiment, wherein a rotation in the x, y-plane is performed;

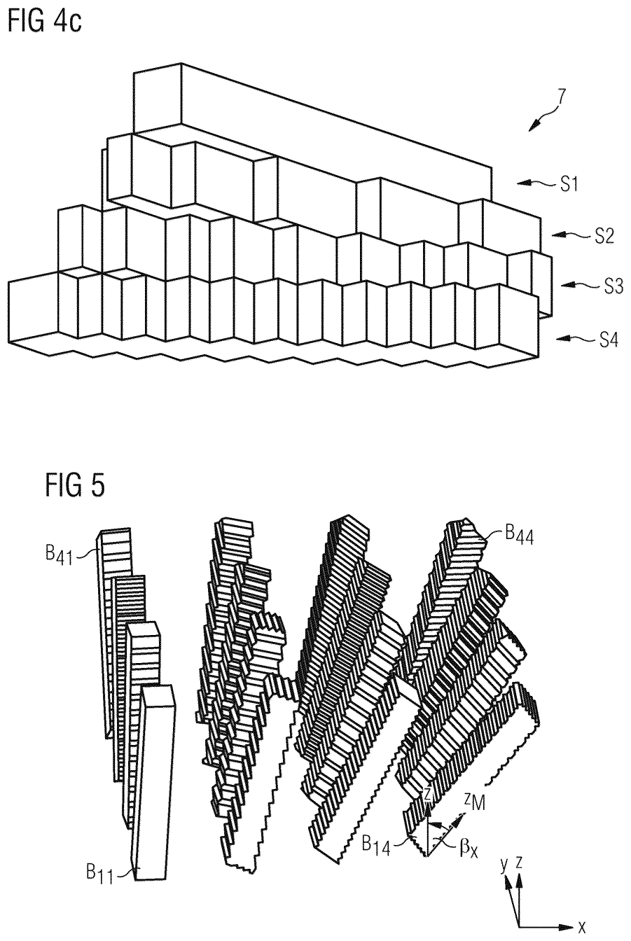

FIG. 5 show simplified views of a rod-shaped component for elucidation of process steps in the selective production of different surface topologies according to a third exemplary embodiment, wherein a tilting relative to the z-axis is performed;

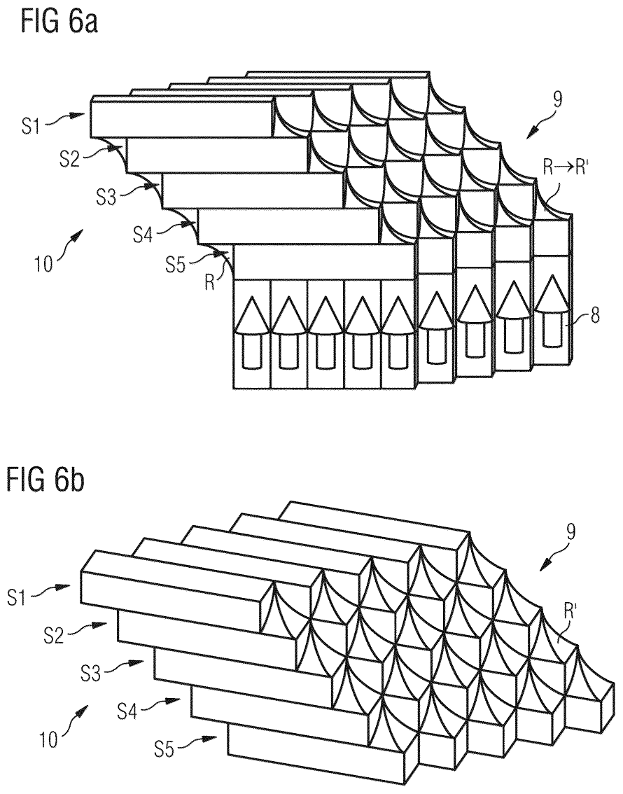

FIGS. 6a to 6c show simplified perspective views for elucidation of process steps according to a fourth exemplary embodiment, wherein different surface topologies are formed simultaneously as a result of a transirradiation on opposite sides; and

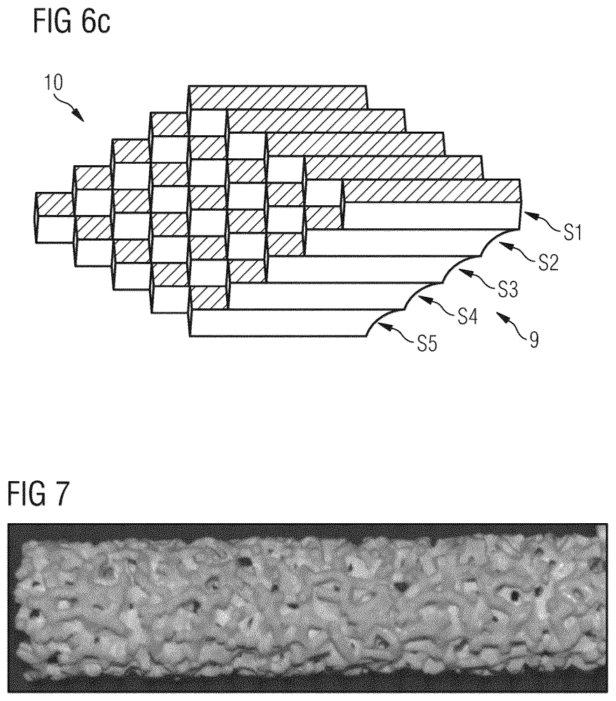

FIG. 7 is a simplified perspective view of a joint implant producible by the process according to the invention for tissue regeneration at a joint.

The subsequent description relates in particular to a process for producing a joint implant for tissue regeneration at a joint. In the same way the process may also be used for producing technical functional materials where wetting behaviour may be adjusted at different surface regions through targeted alteration of surface topologies.

FIG. 1 shows a simplified side view of an apparatus for layerwise buildup of a monolithic component, such as may be used for realizing the process according to the invention. A corresponding apparatus is also referred to as a so-called 3-D printer.

Such a 3-D printer produced for example by Lithoz® is for example based on a lithography-based manufacturing technique, wherein starting from a CAD model of the component to be manufactured the corresponding data sets are directly transmitted to a control unit of the 3-D printer (not shown).

A photopolymerizable material 1 may for example be introduced into a tray having a transparent horizontal tray bottom 2 and uniformly distributed in the tray for example with a height adjustable smoothing element/doctor blade 3. The employed photopolymerizable material 1 may be for example a ceramic suspension (particle-laden dispersion, also known as slip). A platform 4 movable in the Z-direction (vertical) is immersed into the photopolymerizable material 1 to a predetermined distance to the tray bottom 2 before a selective irradiation of the photopolymerizable material 1 is performed to form a polymerized 3D layer body adherent to the platform. The height of the polymerized layer body corresponds to the predetermined distance of the platform 4 from the tray bottom 2, while its base area corresponds to a respective irradiation area. The selective irradiation may therefore be understood as meaning in particular a site-selective irradiation.

As per FIG. 1 the selective irradiation of the photopolymerizable material 1 may be performed from below through the transparent tray bottom 2 using an irradiation unit 5. Blue light having a wavelength of about 465 nm may be used for example. The selective irradiation allows the photopolymerizable material 1 to be polymerized (crosslinked/consolidated) and formed as the first polymerized layer body adherent to the platform 4 of a monolithic component/green body 7 to be produced.

The platform 4 comprising the first polymerized layer body may subsequently be displaced vertically upwards in the Z-direction, photopolymerizable material 1 may optionally be replenished as required and optionally smoothed/uniformly distributed with the smoothing element 3 and the platform finally re-immersed into the photopolymerizable material 1 to a predetermined distance of the previously polymerized layer body to the tray bottom 2 and (site-) selectively irradiated to form a further polymerized layer body. Repetition of these layer production steps finally forms the desired monolithic and three-dimensional component 7 (green body) from a multiplicity of polymerized and mutually adherent layer bodies, wherein a surface of the component in the Z-direction may at least partially have a stepped structure/topology.

As per FIG. 1 the irradiation unit 5 may comprise a digital micromirror unit 6 which allows selective irradiation of the photopolymerizable material 1.

FIG. 2a shows a simplified view of a micromirror unit 6 that is employed for example and FIG. 2b shows a simplified view of a section of an accompanying digital micromirror device (DMD), such as may be employed according to the invention.

As per FIGS. 2a and 2b the micromirror device may comprise a multiplicity of swivellable micromirrors M arranged in rows and columns which may be irradiated by a light source/radiation source L. The micromirrors M may be controlled via a control unit (not shown) such that the incident light (1st beam from the right) is either reflected in the direction of the platform 4 for realization of a pixel P and for crosslinking of the photopolymerizable material 1 present there (2nd beam from the right) or deflected in the direction of an absorber A (no crosslinking of the photopolymerizable material 1 present in the region of the platform 4) (4th beam from the right). In a non-actuated (voltageless) state the light from the swivellable micromirrors M may likewise be deflected in the direction of the absorber A (3rd beam from the right), as a result of which in turn no crosslinking of photopolymerizable material takes place.

In FIG. 2b the micromirror device may comprise about 1000 to 10 000 rows and columns of micromirrors M. A micromirror M may further represent one or more pixels P. For example a micromirror device having a resolution of 4096×2100 pixels (4K) may be used. The micromirrors M may for example be square with a side length of about 10 μm to 20 μm. In FIG. 2b a pixel P actually imaged on the tray bottom 2 may also have a size that is adjustable/alterable via an imaging unit O. The optical imaging unit O is likewise controllable via a control unit (not shown) and may comprise one or more lenses. Alternatively, the micromirrors M of the micromirror device may also assume only two states, wherein the reflected light is either deflected into the absorber A or into the imaging unit O.

The process for layerwise buildup of a monolithic component with the apparatus according to FIGS. 1 and 2 may for example have a resolution of about 10-100 μm in the X-Y direction and a resolution of about 5-100 μm in the Z direction. The maximum 3-D dimensions for the green body 7 to be formed (3-D printing) may be 76×43×150 mm³ for example.

The completion of the additively manufactured 3-D layer body/green body 7 may be followed by a final cleaning, debindering and sintering, thus making it possible to obtain the final monolithic component.

The axes X, Y and Z shown in FIG. 1 characterize the principal axes of the actual build space/manufacturing space and are hereinbelow also referred to as the build space orientation.

By way of example the photopolymerizable material 1 may comprise a polymer, in particular PA, PEK, PEKK, UHMWPE or PCL; a metal, in particular Ti or stainless steel; a metal alloy, in particular Ti64 or CoCr; a magnesium alloy, in particular Mg—Ca, Mg—Zr or Mg—Zn; a ceramic, in particular Al2O3, ZrO2 or Ca3 (PO4) 2; and/or Si3N4. This makes it possible to realize for example a high mechanical strength joint implant having improved properties for tissue regeneration or a technical functional body having improved properties in terms of mechanical wear, use as a sensor, use as a capacitor, use as a catalyst, electrophoresis and/or targeted deposition of materials. The green body may either be used as a particle-filled polymer component or, after subsequent processing, be in the form of a metallic/ceramic component.

It is in principle also possible to employ, especially for production of the joint implant, further medically approved, bioinert and biocompatible as well as 3-D-printable materials for the photopolymerizable material 1.

FIGS. 3a and 3b show simplified perspective views for elucidation of process steps according to a first exemplary embodiment, wherein different surface topologies are formed as a result of a deliberate alteration of a design orientation of a design element relative to a build space orientation.

FIG. 3a for example shows identical design elements E having different design orientations (X_{M}, Y_{M}, Zm_{M}), such as may be produced by a rotation in a design plane (CAD or CAM). A multiplicity of such design elements E may form a design component (not shown).

FIG. 3b shows accompanying actual component elements B such as are producible in a build space (X, Y, Z) by an additive manufacturing technique. As is shown in FIG. 3b for a contemplated design surface O_{M} of the left hand design element E the additive manufacturing technique (layerwise buildup) results in stepped surface topologies for the corresponding component surface O_{B} of the left hand component element B.

According to the invention it is now possible already during the design of the respective design elements E to effect specific alteration of a design orientation (X_{M}, Y_{M}, Z_{M}) such that a different surface topology and thus surface property is realized for the contemplated design surface O_{M} of the design element E. In particular the specific alteration of the design orientation (X_{M}, Y_{M}, Z_{M}) relative to a build space orientation (X, Y, Z) shown in FIG. 3a for the right hand design element E for example can now produce a smooth component surface O_{B} in an actual build space. It is thus possible to realize locally different surface properties in particular in terms of wetting behaviour, deposition behaviour and/or colonization with cells in specifically selected regions of a component to be produced.

FIGS. 4a to 4c show simplified views for elucidation of process steps in the selective production of different surface topologies according to a second exemplary embodiment, wherein a rotation between the horizontal build space principal axes/projected micromirror principal axes X, Y according to FIGS. 2a and 2b relative to the horizontal design element principal axes X_{M}, Y_{M} is performed.

FIG. 4a shows a simplified plan view of a pixel field projectable onto the tray bottom 2 by the micromirror unit 6 which represents the horizontal built space principal axes/micromirror principal axes X, Y. The design element principal axes X_{M}, Y_{M} of the component to be produced are typically aligned parallel and unalterably relative to these micromirror principal axes X, Y (α=0°), thus making it possible for example to form a layer body S1 having straight (smooth) lateral surfaces. Rotation of the design element principal axes X_{M}, Y_{M} relative to the micromirror principal axes X, Y by a rotation angle x now makes it possible to form different surface topologies on the lateral surfaces of the formed layer bodies S2 to S4, as shown in FIG. 4a.

As per FIG. 4a it is thus possible at a rotation angle between the micromirror principal axes X_{M}, Y_{M} (horizontal build space principal axes) relative to the design element principal axes X, Y of α=45° to form a surface topology on the lateral surfaces for a layer body S4 which is substantially defined by the dimensions of the pixels P projected onto the tray bottom 2.

FIG. 4b shows an accompanying perspective view of the plan view shown in FIG. 4a. As is apparent from FIG. 4c a successive layer buildup of the layer bodies S1 to S4 with alteration of the rotation angle α of 0° to 45° makes it possible to produce a 3-D body 7 which has smooth lateral surfaces in its upper region (layer body S1) while its lowermost region (layer body S4) has a very rough (stepped) lateral surface.

Thus, according to this second exemplary embodiment alteration of a rotation angle α between the horizontal design element principal axes X_{M}, Y_{M} and a projected row/column alignment X, Y of the micromirror unit 6 during the repeating of the layer production steps allows specific alteration of a surface topology of the monolithic component 7, thus making it possible to realize for example different wetting behaviour in different local regions.

FIG. 5 shows simplified views of resulting 3-D layer bodies for elucidation of process steps in the selective production of different surface topologies according to a third exemplary embodiment, wherein a tilting between a vertical design element principal axis Z_{M} and a vertical build space principal axis Z/the perpendicular to the tray bottom 2 is performed.

In FIG. 5, X, Y describe the horizontal principal axes of a horizontal tray bottom 2 and Z describes a perpendicular or vertical to this horizontal tray bottom 2. Z_{M} describes a vertical design element principal axis, along which the respective layer bodies are formed as described hereinabove.

FIG. 5 shows as the component a rod having 16 different orientations to the platform/to the vertical principal axis of the build space. The vertical principal axis Z_{M} of the design component/rod B₁₁ is parallel to the perpendicular Z to the tray bottom. The rods B₁₂ to B₁₄ are inclined in the XZ-plane while the rods B₂₁ to B₄₁ are inclined in the YZ plane. The further rods B₂₂ to B₄₄ shown in FIG. 5a are inclined both in the XZ-plane and in the YZ-plane.

With respect to the horizontal principal axes of the tray bottom the design element principal axis Z_{M} may be tilted by a tilting angle βₓ in the X-direction and by a tilting angle β_{y} in the Y-direction. Tilting both in the X-direction and in the Y-direction may also take place (β_{x,y}).

FIG. 5 shows a monolithic component B₁₁ formed with a tilting angle of βₓ=0 and β_{y}=0, as a result of which the four lateral surfaces of the component exhibit no stepped surface topologies.

In FIG. 5 a first row shows three further monolithic components, wherein a tilting angle βₓ in the X-direction is increased in each case. Due to the layerwise construction and the tilting in the X-direction a tilting angle of βₓ=45° results in the monolithic component labelled B₁₄ in FIG. 5a which exhibits a stepped surface topology on its two lateral surfaces inclined/tilted in the X-direction while the lateral surfaces not inclined/tilted in the Y-direction have a smooth surface.

Similarly, FIG. 5 in a first column shows three further monolithic components, wherein a tilting angle β_{y} in the Y-direction is increased in each case. Due to the layerwise construction and the tilting in the Y-direction a tilting angle of β_{y}=45° in turn results in the monolithic component labelled B₄₁ in FIG. 5 which exhibits a stepped surface topology on its two lateral surfaces inclined/tilted in the Y-direction while the two lateral surfaces not inclined/tilted in the X-direction have a smooth surface.

Similarly, FIG. 5 in further rows and columns shows further monolithic components, wherein a tilting angle βₓ in the X-direction and β_{y} in the Y-direction is increased in each case. The layerwise construction and the tilting in the X- and Y-direction results for example at a tilting angle of βₓ=45° and β_{y}=45° in the monolithic component labelled B₄₄ in FIG. 5 which exhibits stepped surface topologies on each of its four lateral surfaces inclined/tilted in the X- and Y-direction.

A tilting of the vertical design element principal axis Z_{M} relative to a vertical build space principal axis z/the perpendicular to the horizontal tray bottom 2 thus also allows specific alteration of the stepped surface topologies, thus in turn making it possible to specifically adjust wetting behaviour at the respective surfaces.

As a manufacturing parameter it is further possible to alter the distance between the platform/the last polymerized layer body and the tray bottom during the repeating of the layer production steps. This makes it possible through alteration of a height of the respective layer bodies in the vertical direction to form further different surface topologies.

As a manufacturing parameter it is further possible to alter the dimensions of the pixels reflected by the micromirrors M of the micromirror unit 6 via the imaging unit O during the repeating of the layer production steps. This makes it possible to further alter the surface topologies.

FIGS. 6a to 6c show simplified perspective views for elucidation of process steps according to a first exemplary embodiment, wherein a transirradiation makes it possible to simultaneously form different surface topologies on opposite surface sides.

For example a 3-D body having the stepped structure shown in FIG. 6a may be formed in typical fashion by successive formation of a multiplicity of polymerized layer bodies S1 to S5. Platform 4 is not shown in FIG. 3a and would be above the first layer body S1. As shown in FIG. 6a this successive layerwise buildup results in each case in photopolymerizable residual material R remaining in the corners and/or edges of a resulting stepped structure.

According to the present invention this side effect can be utilized in such a way that an illumination parameter is altered (for example increased) such that during selective irradiation (8) for crosslinking of a respective layer body the photopolymerizable residual material R remaining in the corners and/or edges of the transirradiation side of the stepped structure (9) is irradiated through a layer body disposed therebelow (for example S5) and is likewise converted into photopolymerized/crosslinked residual material (R->R′). By contrast, the residual material R remaining on the incident light-side stepped structure (10) is not irradiated (micromirrors M reflect the light into the absorber A), as a result of which no polymerization/crosslinking of the residual material R occurs in this region. This uncrosslinked residual material may be partially or completely removed in the subsequent cleaning.

As per FIGS. 6b and 6c this results in a relatively rough surface topology for the incident light-side stepped structure (10) and in a surface topology smoothed by the crosslinked residual material R′ for the transirradiation-side stepped structure (9) (corners and/or edges are filled with polymerized residual material R′), thus making it possible to realize for example different wetting behaviour in these local regions.

An irradiation parameter that may be changed for example is an irradiation intensity, wherein an energy input of the radiation into the respective lowermost layer bodies S5 (to be formed) is increased such that the photopolymerizable residual material R disposed thereabove is also (trans) irradiated. As a result, not only the layer body S5 that is actually to be formed but also residual material in a layer plane disposed thereabove is polymerized/crosslinked, with the result that after a cleaning step the depicted smoothing of the transirradiation-side stepped structure 9 is attained. However, since no irradiation from below is performed in the region of the incident light-side stepped structure 10, no postcrosslinking of the photopolymerizable residual material R takes place on this side, with the result that after a cleaning step a very sharp-edged surface topology is formed on the incident light side. By way of example the irradiation intensity may be increased by a factor of 1.5 to 2.5 relative to an irradiation intensity normal for a predetermined layer thickness.

Alternatively or in addition it is possible to increase the irradiation parameter irradiation time, wherein an energy input of the radiation into the lowermost layer body S5 (to be formed) is in turn increased such that the residual material R disposed thereabove is also (trans) irradiated. As a result, not only the layer body S5 that is actually to be formed but also photopolymerizable residual material in a layer disposed thereabove is polymerized/crosslinked, with the result that the depicted smoothing is attained on the transirradiation-side stepped structure 9 on account of the polymerized residual material R′. No irradiation from below is in turn performed in the region of the incident light-side stepped structure 10, as a result of which no postcrosslinking of the polymerizable residual material R takes place on this side and (for example in the case of a cleaning of the uncrosslinked residual material R removed) a very sharp-edged surface topology is formed. By way of example the irradiation time may be increased by a factor of 1.5 to 3.5, preferably a factor of 2, relative to an irradiation time normal for a predetermined layer thickness.

Alternatively or in addition it is furthermore possible to alter a wavelength of the radiation used to increase an energy input and realize the effect described hereinabove.

Although the present application often refers to light in respect of the irradiation parameters it will be appreciated that any type of electromagnetic radiation (also including for example invisible light, x-rays etc.) may be used for the selective irradiation.

This first exemplary embodiment accordingly makes it possible to form different surface topologies on opposite sides of a 3D body/monolithic component in individual surface regions in particularly simple fashion and especially simultaneously through specific variation of irradiation parameters.

FIG. 7 shows a simplified perspective view of a joint implant producible by the process according to the invention for tissue regeneration at a joint.

As per FIG. 6 an artificial trabecular structure may be formed at least in a shell region, wherein a surface topology of the respective trabecula may be specifically altered with the process described hereinabove. The trabecular structure of the joint implant which is open and permeable to bodily fluids in principle allows rapid colonization of the trabecular surface with cells such as chondroblasts or osteoblasts. Furthermore the specific alteration of the surface topology (not visible) described above may be used to afford a joint implant, wherein growth of a regenerative fibrocartilage up to a high-quality, hyaline regenerative cartilage (chondroblasts) is favoured in certain regions and bone growth (osteoblasts) is favoured in other regions.

The joint implant preferably has a length of at least 0.6 cm and at most 1.2 cm for patellar applications and applications in small joints such as for example the wrist or ankle and at least 0.8 cm and at most 2.2 cm, in particular 1.0 cm to 1.6 cm and more preferably 1.25 cm for respective proximal and distal tibial and femural application in the knee and hip joints. This makes it possible to achieve optimal accessibility and ingrowth of mesenchymal stem cells. The joint implant according to FIG. 7 may further have a diameter D of at least 2 mm and at most 6 mm, preferably 3 mm, which makes it possible to achieve an optimum of lateral surface facing the synovia (joint cavity) for formation of replacement cartilage tissue. A thickness d of the artificial trabecular structure is preferably 0.5 to 2.0 mm, more preferably 0.5 to 1.5 mm.

A network structure of the artificial trabecular structure which is defined, repetitive and based on the natural trabecular structure or a channel structure with an appropriately adapted shape of the joint implant, in particular in conjunction with the above-described process for forming different surface topologies in specifically selected regions, makes it possible to achieve optimal ingrowth of endogenous tissue into the boundary volume between the joint implant and a depression/bore channel, in particular into the internal volume of the joint implant and above the end of the joint implant facing the synovia.

According to the invention a joint implant may thus in particular have a hydrophobic and thus chondroblastic differentiation-favouring surface in its top region and upper shell region while the bottom region and the lower part of the shell region have a hydrophilic surface to favour an osteoblastic differentiation of mesenchymal stem cells. This makes it possible to favour cartilage growth in the upper region of the joint implant (facing the synovia) and bone growth in the lower region of the joint implant (facing the bone marrow space).

It is alternatively possible to produce technical functional bodies, for example catalysts, capacitors, membranes etc., as such monolithic components having locally different surface properties. On account of a selectively adjustable wetting behaviour technical functional bodies (3-D bodies) can thus be coated specifically and only for defined regions, for example with electrically conductive material, with catalytically active material, with wear-resistant material, with sensor material, with capacitor material etc. Highly complex and very small functional bodies are thus producible at relatively low cost for the first time.

It is noted that each step of the process described hereinabove may be implemented by computer program commands. These computer program commands may be loaded onto a computer or another programmable device to produce an apparatus, wherein the commands executed in the computer or another programmable device produce means for implementing the functionalities as described in the process steps. These computer program commands may likewise be stored on a digital storage medium, for example a DVD, CD or diskette comprised by a computer or other programmable device for realizing a certain functionality. Furthermore, the computer program commands/the program code may be downloaded for example from a telecommunications network to bring about operating steps which are implemented on a computer or another programmable device to produce a computer-implemented process which makes it possible to perform the process steps.

The invention thus further comprises a digital storage medium with electronically readable control signals which can interact with a computer system such that they can execute the described process steps. The invention further relates to a computer program product comprising program code stored on a machine-readable storage medium for performing the described process steps when the program is executed on a computer. The present invention further relates to a computer program comprising program code for performing the process steps described hereinabove when the program is executed on a computer.

The invention was described hereinabove with reference to preferred exemplary embodiments. However, said invention is not limited thereto and in particular also comprises combinations of the above-described exemplary embodiments. In particular, an irradiation may also be effected via lasers instead of the micromirror unit. It is also possible to employ other additive 3D printing processes instead of the described lithography process.

### LIST OF REFERENCE NUMERALS

1 photopolymerizable material
2 tray bottom
3 smoothing element
4 platform
5 irradiation unit
6 micromirror unit
7 monolithic component (green body)
8 selective irradiation
9 transirradiation-side stepped structure
10 incident light-side stepped structure
11 pixel field
12 lifting mechanism
S1-S5 layer body
M micromirror
L light source
A absorber
P pixel
O optical imaging unit
E design element
O_{M} design element surface
B component element
O_{B} component element surface
R photopolymerizable residual material
R′ photopolymerizable residual material
X, Y horizontal build space orientation (micromirror principal axes)
X_{M}, Y_{M}, Z_{M} design element orientation
Z vertical build space orientation (perpendicular to tray bottom)
α rotation angle
βₓ tilting angle in x-direction
β_{y} tilting angle in y-direction
B₁₁-B₄₄ components produced with tilting

We claim:

1. A process for producing a monolithic component, wherein different surface topologies for realizing different surface properties, in particular in terms of wetting behaviour, deposition behaviour and/or colonization with cells, are produced in specifically selected regions.

2. The process according to claim 1, wherein the monolithic component is produced from a component design (CAD) having a multiplicity of design elements (E), wherein at least one design orientation (X_{M}, Y_{M}, Z_{M}) of a design element (E) relative to a build space orientation (X, Y, Z) of an actual build space (X, Y, Z) is specifically altered.

3. The process according to claim 1 comprising the steps of:
forming the monolithic component using an additive manufacturing technique, wherein a photopolymerizable material (1) is provided in a tray having a transparent horizontal tray bottom (2),
a platform (4) is immersed in the photopolymerizable material (1) to a predetermined distance from the tray bottom (2), a selective irradiation (8) through the tray bottom (2) is performed to produce a polymerized layer body (Sₓ) adherent to the platform (4) which has a height corresponding to the distance,
the platform (4) comprising the polymerized layer body (Sₓ) is vertically displaced to form a subsequent layer body (Sₓ₊₁) and
by repeating the preceding layer production steps the monolithic component (7) is built up layerwise in the desired shape, wherein a surface of the component at least partially has a stepped structure,
wherein
adjusting manufacturing parameters during the repeating of the layer production steps produces different surface topologies for realizing different surface properties in selected regions of the monolithic component (7).

4. The process according to claim 3,
wherein the selective irradiation (8) is carried out using a digital micromirror unit (6) comprising a multiplicity of micromirrors (M) arranged in rows and columns.

5. The process according to claim 3,
wherein
as a manufacturing parameter an irradiation parameter, in particular an irradiation intensity and/or an irradiation time, is in the selective irradiation (8) through the tray bottom (2) adjusted such that simultaneously with the layer body to be polymerized a photopolymerizable residual material (R) remaining in the edges and/or corners of a step structure disposed thereabove is polymerized.

6. The process according to claim 3, wherein as a manufacturing parameter a rotation angle (α) of horizontal micromirror principal axes (X, Y) relative to horizontal design element principal axes (X_{M}, Y_{M}) is altered during the repeating of the layer production steps.

7. The process according to claim 3, wherein as a manufacturing parameter a tilting angle (B) between a vertical design element principal axis (Z_{M}) and a perpendicular (Z) to the horizontal tray bottom is altered during the repeating of the layer production steps.

8. The process according to claim 3, wherein as a manufacturing parameter the distance between the platform/the last polymerized layer body (Sₓ) and the tray bottom (2) is altered during the repeating of the layer production steps.

9. The process according to claim 4, wherein as a manufacturing parameter the dimensions of the pixels (P) reflected by the micromirrors (M) of the micromirror unit are altered via an imaging unit (O) during the repeating of the layer production steps.

10. The process according to claim 3, wherein the photopolymerizable material comprises:
a material from the class of polymers, in particular PA, PEK, PEKK, UHMWPE or PCL,
a material from the class of metals, in particular Ti or stainless steel;
a material from the class of metal alloys, in particular Ti64 or CoCr,
a material from the class of magnesium alloys, in particular Mg—Ca, Mg—Zr or Mg—Zn,
a material from the class of ceramics, in particular Al₂O₃, ZrO₂, Si₃N₄ or Ca₃(PO₄)₂,
and/or
a material from the class of glasses.

11. The process according to claim 3, wherein the production of the monolithic component (7) is followed by a cleaning, debindering and/or sintering.

12. The process according to claim 1, wherein the monolithic component (7) is a joint implant for tissue regeneration at the joint.

13. The process according to claim 1, wherein the monolithic component (7) is a technical functional body.

14. A digital storage medium with electronically readable control signals which can interact with a computer system such that a process according to claim 1 is executed.

15. A computer program product comprising non-transitory program code stored on a machine-readable storage medium for performing the process according to claim 1 when the program is executed on a computer.

16. The computer program comprising program code for performing the process according to claim 1 when the program is executed on a computer.
